(19) United States
(12) Patent Application Publication
(10) Pub. No.: US 2007/0225740 A1
(43) Pub. Date: Sep. 27, 2007

(54) Endoscopic Pulley Knife Instrument for Transecting Ligaments or Fascia

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/677,650

(22) Filed: Feb. 22, 2007

Related U.S. Application Data
(60) Provisional application No. 60/775,354, filed on Feb. 22, 2006.

(51) Int. Cl. A61B 17/32

(52) U.S. Cl. 606/170

(57) ABSTRACT

An instrument adapted to be placed over the tip of an endoscope has a rearward-facing blade which can be deployed by manipulating an actuator. The actuator is connected to the blade by a flexible tensile member like a cable or wire, which enables one to make and use curved or bendable versions of the instrument. Once the blade is deployed, tissues may be cut by drawing the instrument past the tissues.

This application claims benefit of provisional U.S. patent application No. 60/775354, filed Feb. 22, 2006.

### BACKGROUND OF THE INVENTION

This invention relates to surgery and more particularly to the endoscopic percutaneous transection of ligaments and fascia.

Thickening of ligaments and fascia through repetitive use and aging can give rise to a number of pathological conditions that afflict mankind. Most notably among these is carpal tunnel syndrome, in which the transverse carpal ligament in the hand thickens, compressing the underlying median nerve and causes varying degrees of pain, paresthesias and other associated symptoms of neurologic dysfunction. A similar condition is seen in the foot at the level of the tarsal tunnel and is referred to as the tarsal tunnel syndrome. Thickening of the plantar fascia in the foot can also occur giving rise to heel spur syndrome.

In ischemic conditions, fascia may need to be cut to improve blood flow to affected limbs. The surgical treatment of these conditions requires division of the offending ligament or fascia so that relief of symptoms occurs.

Because the overlying skin and adjacent soft tissues contain various sensory corpuscles and free nerve endings, and because structures beneath the fascia and ligaments often include vital tendons and nerves, division of the offending ligament or fascia with minimal disruption of surrounding tissues is preferred.

Of the minimally invasive techniques employed, an endoscopic uniportal technique seems best fit to subserve the essentials of a single small skin incision with minimal disruption of essential and non pathologic adjacent tissues.

What is needed is an endoscopic pulley knife instrument and method for endoscopic uniportal percutaneous transection of ligaments or fascia that minimizes the risk of damaging adjacent tissues.

### SUMMARY OF THE INVENTION

An object of the invention is to provide a tool which can be mounted on a conventional endoscope that can be passed underneath ligaments or fascia, where the tool has a movable blade that can then be deployed to cut the ligaments or fascia on the backstroke as the endoscope is withdrawn.

The invention enables one to perform an endoscopic minimally invasive method of surgically dividing pathologic ligaments or fascia so that pathologic conditions or consequences can be alleviated mitigated, cured or relieved.

The invention provides a tool for accomplishing surgery through a single skin incision, enabling techniques which qualify both as minimally invasive and as uniportal.

The invention provides an instrument for achieving the above that is compatible with presently available endoscopic systems, enabling hospitals and surgical centers to contain costs.

While the invention is described in reference to the treatment of the most common of such conditions, carpal tunnel syndrome, it should be understood that the instrument and technique described herein are adaptable to many pathologic states in which ligaments or fascia have become pathologically thickened or altered.

In practicing this invention, one or more special endoscopic sheaths of a size and configuration capable of being slipped over the barrel of any present day endoscope may be employed. Embedded at the distal end of the sheath is a cutting blade that can be variably extended or retracted by a pulley system; thus the length of the exposed cutting surface can be controlled with a high degree of accuracy.

The blade is controlled by a string, cable or wire running lengthwise of the sheath, either within a groove on the sheath or simply exposed on the surface of the sheath. Alternatively, a dual sheath mechanism, or a sheath within a sheath, may be employed whereupon the activating wire may be run between the inner and outer sheaths.

The phrase “flexible tension member” as used herein means a cable, string, wire, or other very flexible elongate member having good tensile strength.

In the preferred embodiment, the sheath is slipped over an endoscope with the cutting blade in a retracted state. The endoscope is then inserted beneath the ligament or fascia to be transected. Sufficient tension applied to the wire deploys the pulley knife. The endoscope and sheath then are withdrawn slowly so that the knife incises the ligament as the sheath and the endoscope are withdrawn. Because the endoscope moves in unison with the knife blade, the entire process can be viewed directly by the surgeon. After the ligaments or fascia are transected, the blade is deactivated or retracted and the endoscope and the sheath are removed.

The cutting blade can be deployed and retracted in either a single and dual pulley version. In the former version, the knife blade, normally held in a retracted position by a small spring, is deployed by applying tension to the wire, string or cable. When tension is released, the spring retracts the blade so as to protect surrounding tissues.

In the secondary version, a two-pulley mechanism is employed. Here, there is no need for a return spring: apply tension on one limb of the pulley deploys the blade, while tension on the other limb of the pulley retracts the blade.

A wire pulley system is better than activator shaft required in U.S. Pat. No. 5,306,284, because wire can be very thin, and thus be used in smaller devices, whereas an activator shaft must have sufficient cross section to resist buckling when pushed.

A wire pulley system is superior to the side-by-side moveable sections disclosed in U.S. Pat. No. 5,769,865, whose balloon-activated system is more complex and pushes it blade out laterally against the ligament, rather than drawing the blade against the ligament, like the most effective scalpel techniques.

Other features and advantages of the invention will become apparent from the following description of the preferred embodiments, which refers to the accompanying drawings.

### BRIEF DESCRIPTION OF THE DRAWINGS

The invention is described in detail below with reference to the following figures, throughout which similar reference characters denote corresponding features consistently, wherein:

FIG. 1 is an isometric view of a tool embodying the invention, where the blade of the instrument is contained within a sleeve, in a configuration suitable for inserting the tool through a skin portal and beneath a ligament;

FIG. 2 shows the tool with the sleeve slightly retracted;

FIG. 3 shows the blade free of the sleeve;

FIG. 4 shows the blade in a deployed position, cause by pulling on a handle at the proximal end of the instrument;

FIGS. 5-7 show and alternative form of the invention, in which a lever is raised to deploy the blade;

FIGS. 8-13 show another form of the invention, in which a lever is lifted to raise the blade, against the action of a return spring;

FIG. 14 shows a variation of the invention in which the blade is supported at one end of a flexible sheath, which can be placed over a curved endoscope;

FIG. 15 shows another variation having a return cable, rather than a return spring; and

FIGS. 16-19 show a further embodiment in which the blade is supported by a bearing.

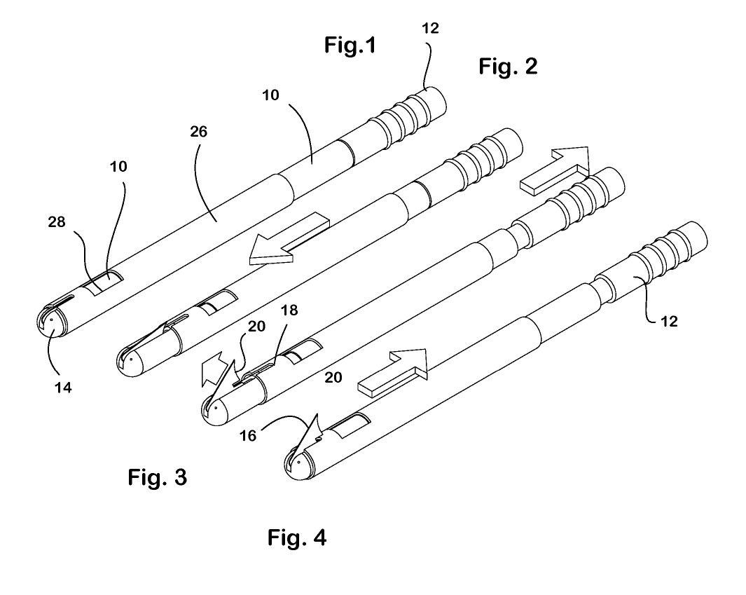

### DETAILED DESCRIPTION OF THE PREFERRED EMBODIMENTS

As shown in FIG. 1, an endoscopic pulley knife embodying the invention includes an elongate body 10 having a telescoping handle 12 at its proximal end, which can be moved axially with respect to the body, (compare FIGS. 3 and 4). The body and the handle are preferably both hollow, so that an endoscope can be inserted through them for purposes discussed below.

The body terminates at a rounded nose 14 preferably made of a soft material designed to prevent tissue damage as it is guided along tissue planes. Just rearward of the nose is a blade 16 mounted on a pivot pin (not shown) so that the blade can move from a lower retracted position (FIG. 3) in which is sits in a groove 18 formed in the body, to a deployed position (FIG. 4) in which its sharp edge 20 protrudes outside the envelope of the body, facing rearward. The blade's path is defined by the arcuate bearing surface 22 and the follower 24, which is secured to the blade. A retractable sleeve 26 normally covers the blade. The handle 12 is connected to the blade by a cable, wire or string (not shown) which passes around a pulley (not shown) in the nose of the tool, ahead of the blade. Once the sleeve is retracted (FIG. 3), the blade can be deployed by pulling on the handle 12 (FIG. 4). Once the blade is deployed, the ligament can be cut by withdrawing the instrument.

The aperture 28 serves as a window so that the surgeon can “see” the cutting action of the blade by looking through the window with a properly positioned side-looking endoscope.

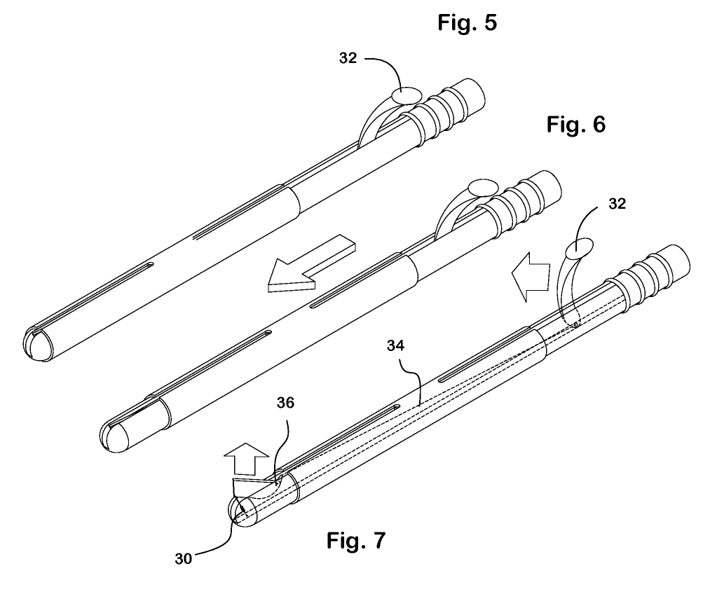

The instrument shown in FIGS. 5-7 works basically the same way. FIG. 7 shows a return spring 30 in the nose of the instrument. The lever 32 at the distal end, when raised (FIG. 9) performs the function of tensioning the cable 34, connected to a tab 36 at the base of the blade, which deploys the blade.

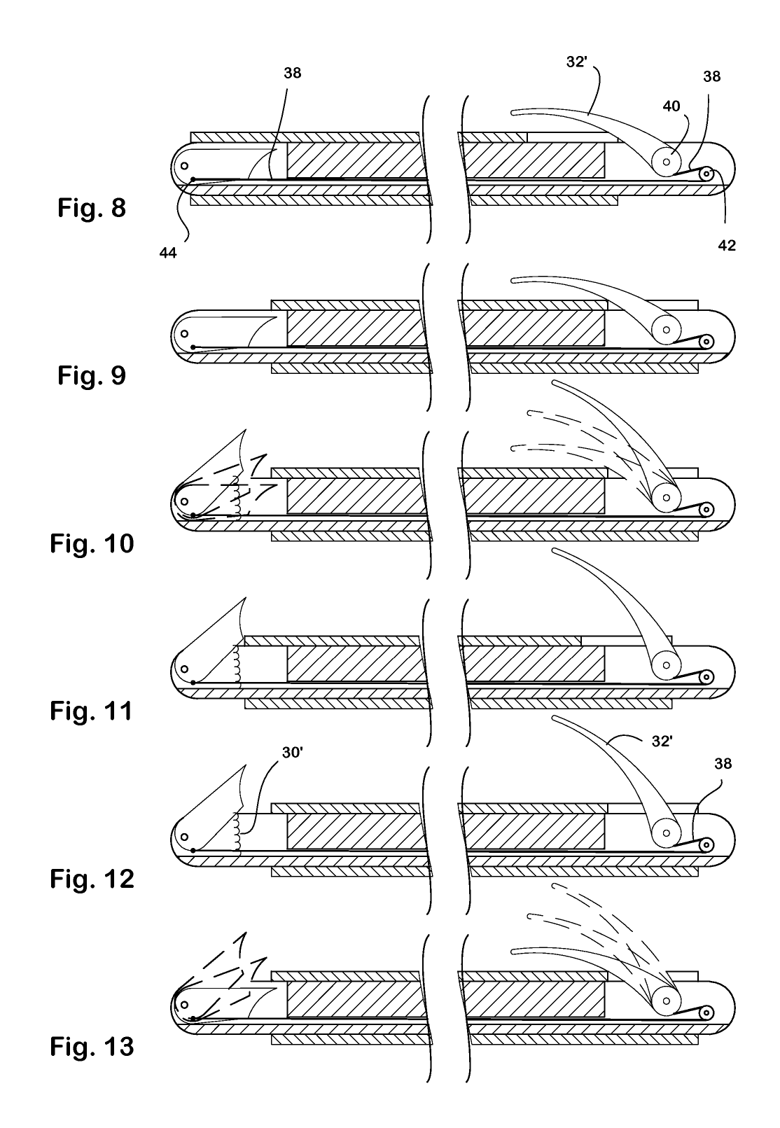

FIGS. 8-13, showing a third embodiment of the invention, one which is not hollow, illustrate the tension cable or wire 38, which winds on the base 40 of the lever 32′ when it is raised. The cable passes around a small idler pulley 42 to the rear of the lever. The distal end of the cable is secured to the blade at a radially offset point 44, so that the tension rotates the blade in a direction opposite to that urged by the spring 30′.

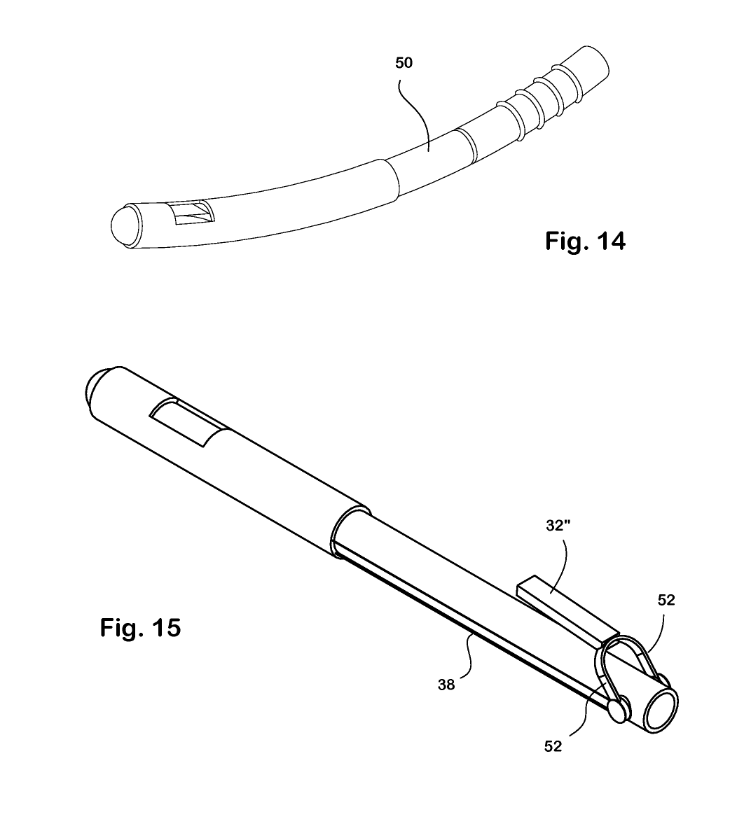

An advantage of this invention is that, because the tension member is flexible, it can be used on curved endoscope tips simply by making the body a flexible sheath adapted to fit over the endoscope tip. FIG. 14 shows one such version, the sheath being identified by numeral 50. Here, the lever 32″ has a forked lower end, each tine 52 being to one side of the centerline of the sheath, so that the inner volume of the sheath is unobstructed and it can be placed over an endoscope tip.

FIG. 15 shows a version of FIG. 14 in which the cable is continuous, like a clothesline, so that no return spring is required. With this arrangement, tension in one run 54 of the cable deploys the blade, while tension in the other run 56 retracts the blade.

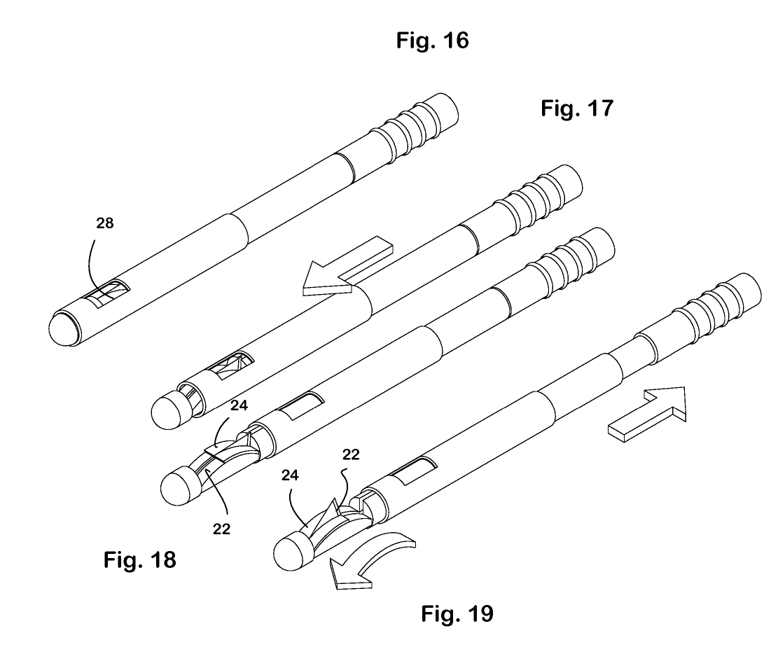

In the embodiment of FIGS. 16-19, the blade is mounted on an arcurate bearing which slides on a conforming support surface to move the blade between a stowed position (FIG. 17) and a deployed position (FIG. 19).

Inasmuch as the invention is subject to modification and variations, the foregoing description and the drawings should be regarded as merely illustrative of the invention defined by the claims below.

I claim:

1. An instrument for percutaneous transection of ligaments or fascia comprising:
a body;
a blade pivotally mounted on the body;
an actuator mounted on the body; and
a flexible tension member interconnecting the blade and the actuator in such a way that inducing tension in the flexible tension member causes the blade to pivot relative to the body from a retracted position to a deployed position.

2. The instrument of claim 1, further comprising a spring biasing the blade toward its retracted position.

3. The instrument of claim 1, wherein the actuator is a lever pivotally mounted on the body.

4. The instrument of claim 1, wherein the actuator is a handle slidingly mounted on the body.

5. The instrument of claim 1, wherein the body is tubular and is configured to receive an endoscope.

6. The instrument of claim 1, wherein the body is curved to facilitate positioning the apparatus relative to a surgical site.

7. The instrument of claim 1, wherein the body is a tubular sheath through which an endoscope can be inserted.

8. The instrument of claim 7, wherein the sheath is substantially flexible so that it can be passed over a curved endoscope tip.

9. The instrument of claim 7, wherein the sheath is configured to allow the actuator complete range of motion when the blade and the sheath are positioned for cutting.

10. The instrument of claim 7, wherein the sheath has a lateral aperture near the blade so that the action of the blade can be viewed through the endoscope.

11. The instrument of claim 1, wherein the tension member runs in a closed loop between the actuator and the blade, so that the tension member deploys the blade when moved in one direction, and retracts the blade when moved in the opposite direction.
